(19) United States
(12) Patent Application Publication
(10) Pub. No.: US 2017/0227473 A1
(43) Pub. Date: Aug. 10, 2017

(54) PHOTOGRAPHIC METHODS OF QUANTIFICATION FOR WATER AND DEBRIS MIXTURES

(71) Applicant: ATOMIC ENERGY OF CANADA LIMITED / ÉNERGIE ATOMIQUE DU CANADA LIMITÉE, Chalk River (CA)

(72) Inventors: David Bruce Rhodes, Deep River (CA); Harry Millan Adams, Deep River (CA)

(21) Appl. No.: 15/320,460

(22) PCT Filed: Jun. 18, 2015
(86) PCT No.: PCT/CA2015/050561

§ 371 Date: Dec. 20, 2016

Related U.S. Application Data
(60) Provisional application No. 62/015,169, filed on Jun. 20, 2014.

(51) Int. Cl. G01N 21/94; G06T 7/62; G06T 7/00; G06T 5/50; G01N 33/18; G01N 15/02

(52) U.S. Cl. CPC G01N 21/94; G01N 33/18; G01N 15/0227; G06T 7/0004; G06T 5/50; G06T 7/62; G06T 2207/30242; G06T 2207/10148; G06T 2207/10061

(57) ABSTRACT

With a water and debris mixture, a method of quantifying debris content may include obtaining at least one image of a sample of the water and debris mixture. The image may be analyzed to quantify the debris content.

### CROSS-REFERENCE TO RELATED APPLICATION

This application claims priority to U.S. Provisional Application No. 62/015,169 filed on Jun. 20, 2014, the entire contents of which are hereby incorporated herein by reference.

### FIELD

The present disclosure relates to techniques for analyzing the fibre and/or particulate content of water and debris mixtures. The present disclosure also relates to nuclear power plant safety.

### BACKGROUND

The following paragraphs are not an admission that anything discussed in them is prior art or part of the knowledge of persons skilled in the art.

In water cooled nuclear power plants, following a loss of coolant accident (LOCA), water and insulation debris dislodged at the break location may accumulate in the sump area. After the initial emergency water injection phase, the sump water may be re-circulated back to the reactor core as part of the emergency core cooling (ECC) system to prevent fuel melt. The debris in the sump water may be filtered by ECC strainers so that the debris will not deposit in the reactor core, which may result in flow blockages and buildup of thermal resistance layers on fuel elements, and may cause the fuel to overheat and melt. Although ECC strainers may catch almost all debris on the strainer surface, a small amount of debris may go through the strainer holes and into the reactor core.

Deposition of the debris in the reactor core is considered a safety issue, because the nuclear fuel keeps producing nuclear energy even after the reactor is safely shutdown (through radioactive decays of unstable isotopes produced in the core). For various water cooled reactors (e.g., PWRs, BWRs and CANDUs), there are either established safe limits of how much debris is allowed to bypass the strainers, or such limits are being developed. Although typical debris includes various types of particulates and fibres, the bypass limits may be set for the amount of fibre only. It is, hence, a functional requirement that ECC strainers be able to filter fibres so that these fibre bypass limits are not exceeded.

There are existing methods to quantify fibre bypass. These methods include collecting water samples at various times downstream of the strainer. The samples may be useful to quantify the transient evolution of the downstream fibre concentration. The samples may be processed by filtering the debris using a fine filter paper, drying the filter paper, and weighing the increase in the weight of filter paper (this technique may be referred to as the “weighing technique”). The overall fibre bypass may then be obtained by integrating the weights of the fibres obtained from individual samples over time. Alternatively, a very fine downstream filter may be used to capture all fibres that bypassed the strainer surface (this technique may be referred to as the “downstream filter technique”). This technique may be more accurate as it gives the total weight of the captured fibres, but may get plugged in some tests that use particulates.

In an actual ECC system, the short-term, high-fibre-concentration flow may last a few hours (corresponding to a few flow turnovers). The requirement for the allowable overall fibre bypass may be defined for one month of ECC operation after the start of the ECC recirculation system. The movement of individual fibres from the debris bed and occasional local collapse of the debris bed may provide a steady supply of a small amount of fibre to the downstream. Hence, even if the downstream fibre concentrations are small, a significant amount of fibre may bypass the strainers after the initial high-fibre-concentration transient. The weighing technique may be a good way of quantifying debris bypass shortly after the ECC recirculation pumps are engaged and a significant amount of debris may be bypassing the clean strainer surface. Most of this debris may end up accumulating on the strainer surface, creating a mat of fibres that may eventually behave like a layer of fine filter on top of the strainer surface. As a result, the fibre concentration downstream of the strainer may eventually become sufficiently small to render the weighing technique impractical (because the weight of the captured fibre becomes a very small fraction of the combined weight of the filter and fibre after filtering).

With either the weighing or downstream filter techniques, the filters may be analyzed using Scanning Electron Microscopy (SEM) to determine the amount of fibre on the filter, and also to quantify the fibre size distribution. Limitations of SEM include that it is generally expensive and the turnaround time tends to be relatively long for processing large amounts of filtered fibre samples. Also, SEM may provide a detailed picture of a section of the filter, but not the whole filter area, and hence some error may be introduced while extrapolating the fibre quantity for the whole filter.

Hence, other techniques, used to quantify fibre bypass for the long-term operation of the ECC system, are desirable.

### INTRODUCTION

The following is intended to introduce the reader to the detailed description that follows and not to define or limit the claimed subject matter.

In an aspect of the present disclosure, a method of quantifying debris content of a water and debris mixture may include: obtaining at least one image of a sample of the water and debris mixture; and analyzing the image to quantify the debris content.

The step of obtaining may include obtaining a plurality of images of the sample. The step of obtaining may include focussing the plurality of images at different cross-sections of the sample. The method may further include combining the plurality of images of the sample to generate a composite image including focussed portions of each image. The step of obtaining may include using a camera to obtain the plurality of images of the sample. The step of obtaining may include varying a distance between the camera and the sample to obtain the plurality of images of the sample. The step of obtaining may include progressively decreasing the distance between the camera and the sample.

The step of analyzing may include using image analysis software. The step of analyzing may include using the image analysis software to measure fibre diameter for a plurality of fibres of the mixture. The step of analyzing may include calculating an average diameter. The step of analyzing may include using the image analysis software to measure fibre length for a plurality of fibres of the mixture. The step of analyzing may include calculating a total fibre length. The step of analyzing may include calculating a total fibre volume for the plurality of fibres. The step of analyzing may include using the image analysis software to measure particle diameter for a plurality of particles of the mixture.

The step of obtaining may include using a digital camera, and the at least one image may be at least one digital image. The method may further include, prior to the step of analyzing, thresholding the at least one image.

Other aspects and features of the teachings disclosed herein will become apparent, to those ordinarily skilled in the art, upon review of the following description of the specific examples of the present disclosure.

### BRIEF DESCRIPTION OF THE DRAWINGS

The drawings included herewith are for illustrating various examples of apparatuses and methods of the present disclosure and are not intended to limit the scope of what is taught in any way. In the drawings:

FIG. 1 shows an exemplary photographic setup for fibre bypass filters;

FIG. 2 is a photograph of a fibre bypass filter;

FIG. 6 shows an exemplary photographic setup for insulation fibres;

FIG. 4 is a schematic diagram of a focus stacking technique;

FIG. 5 is an image of insulation fibres produced by focus stacking;

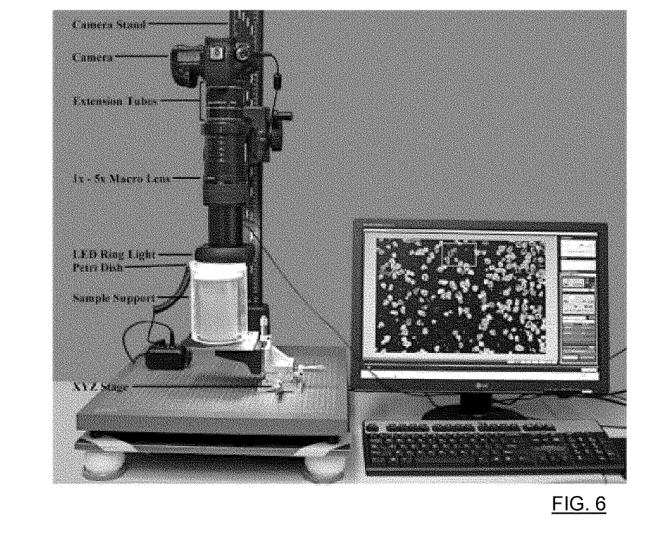

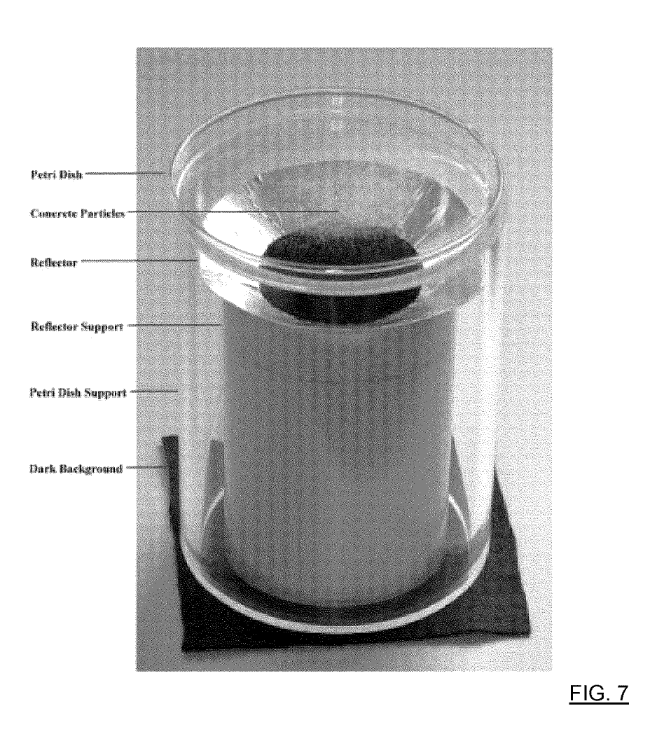

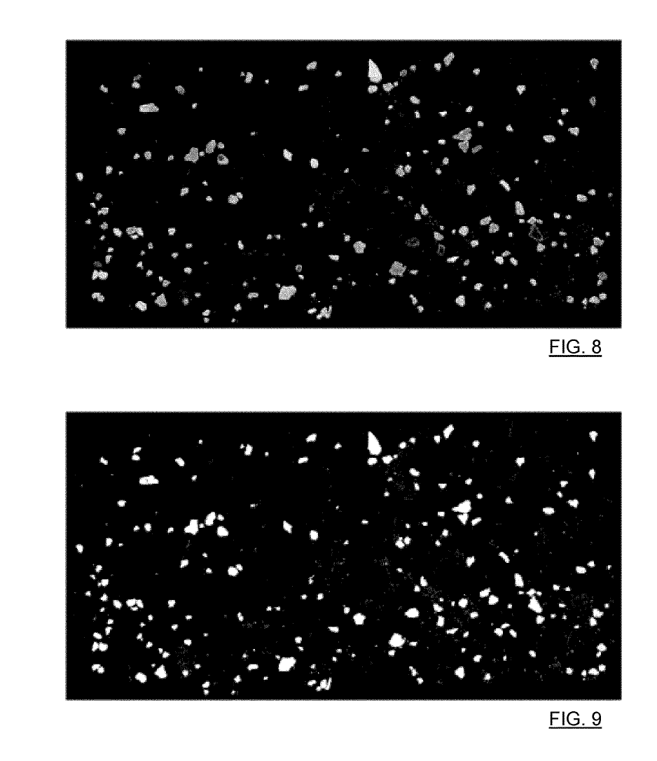

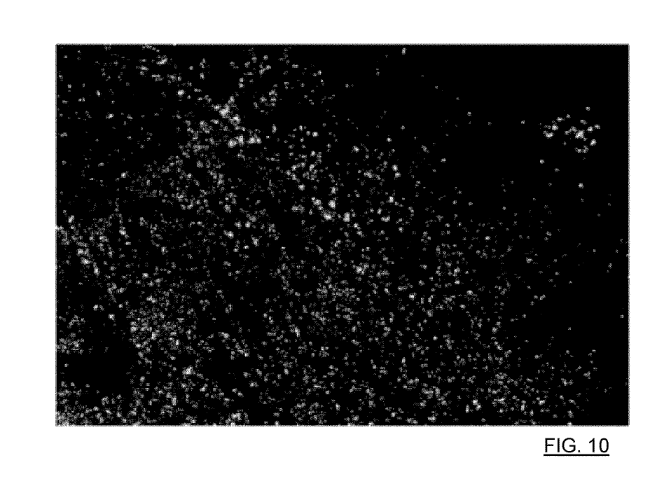

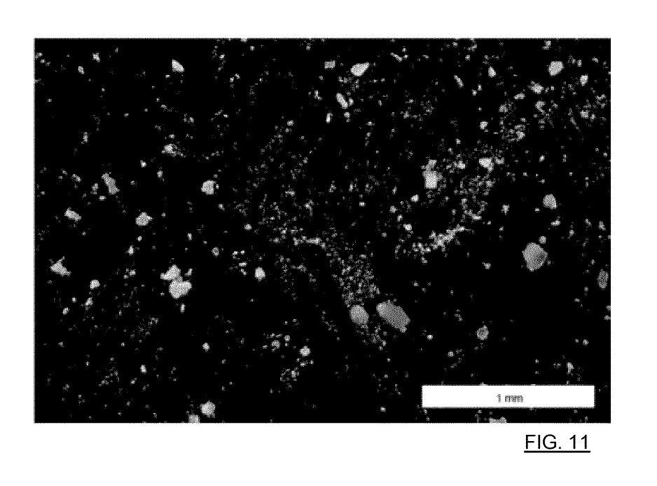

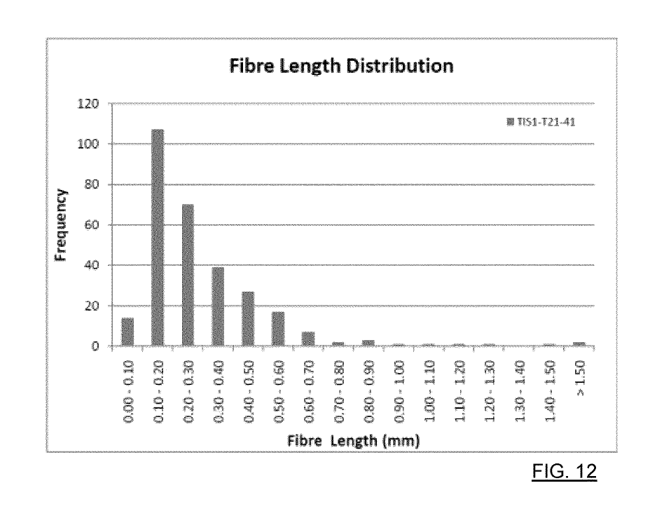

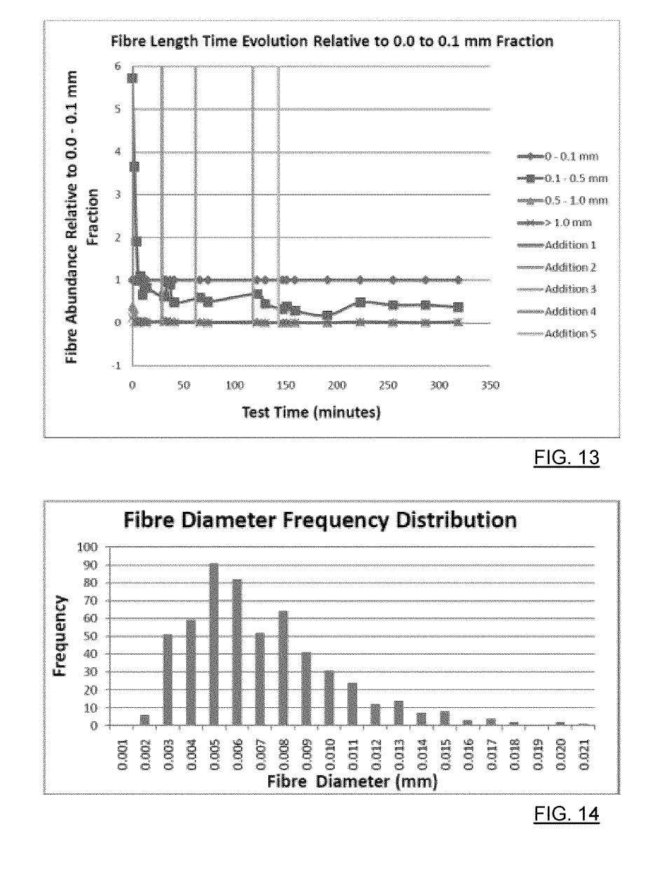

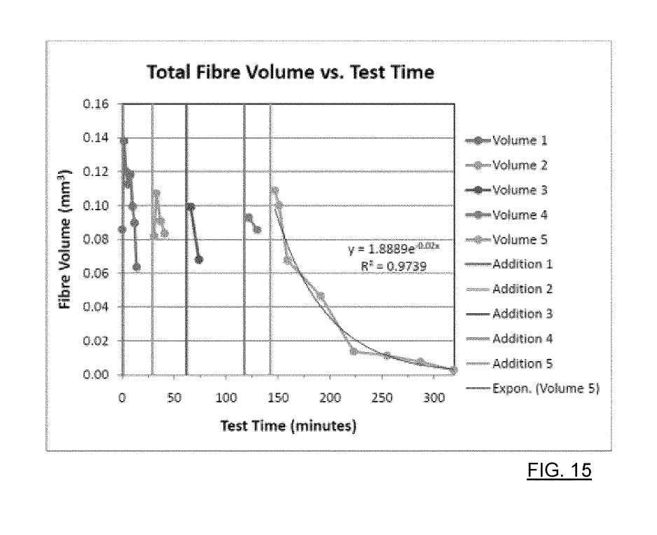

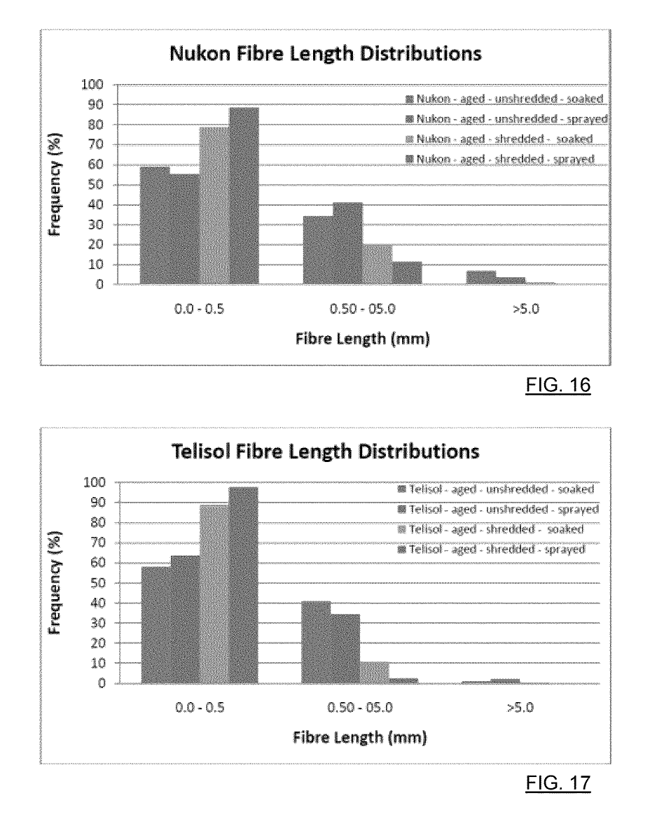

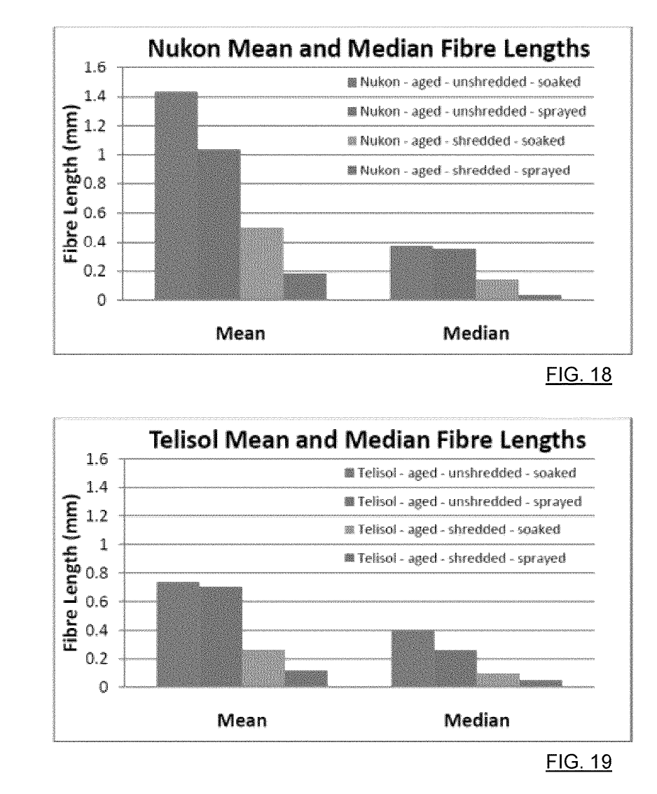

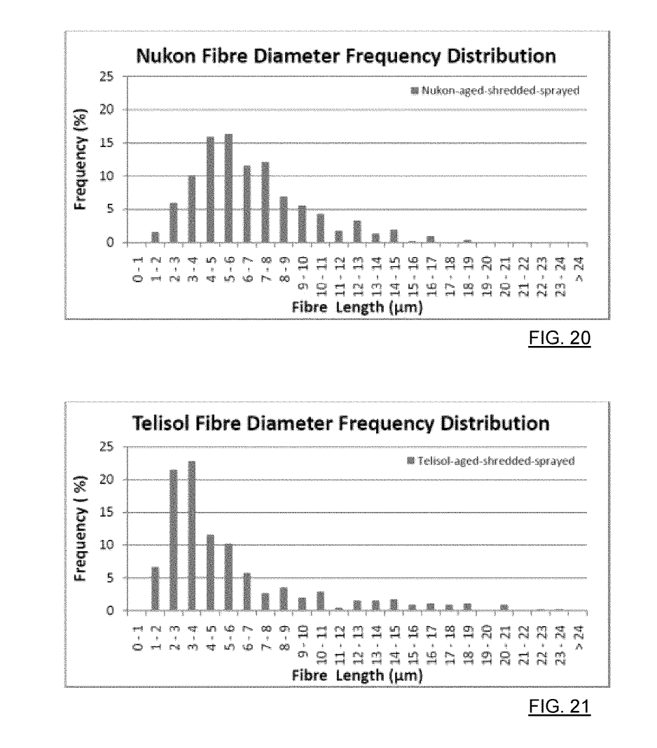

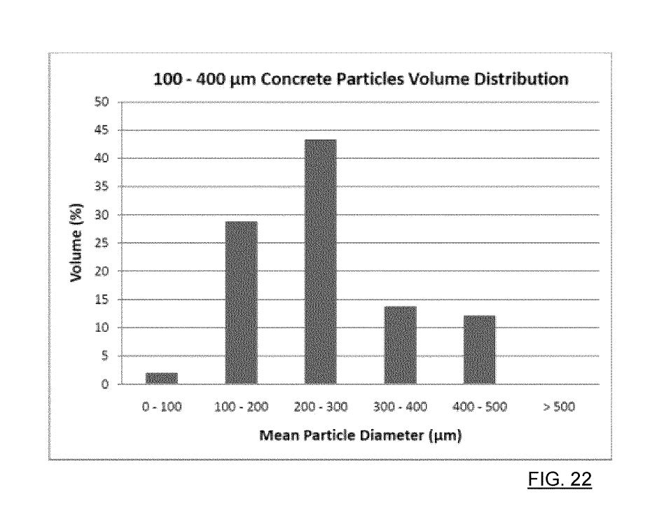

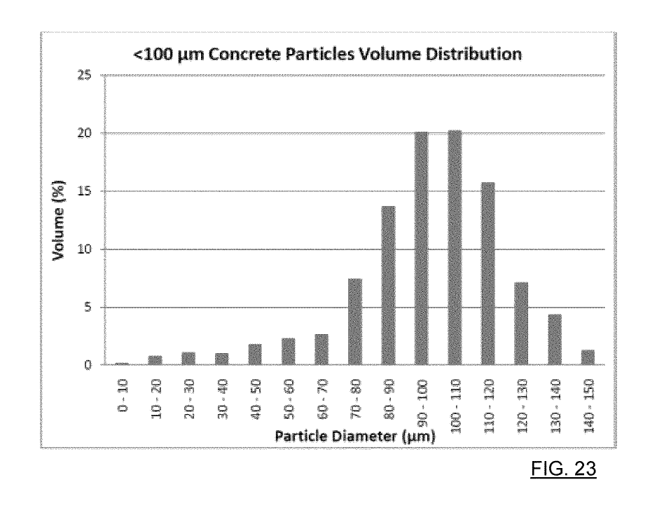

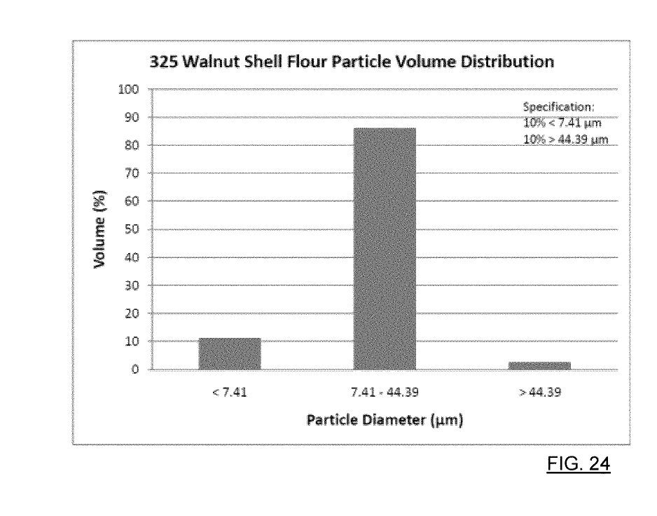

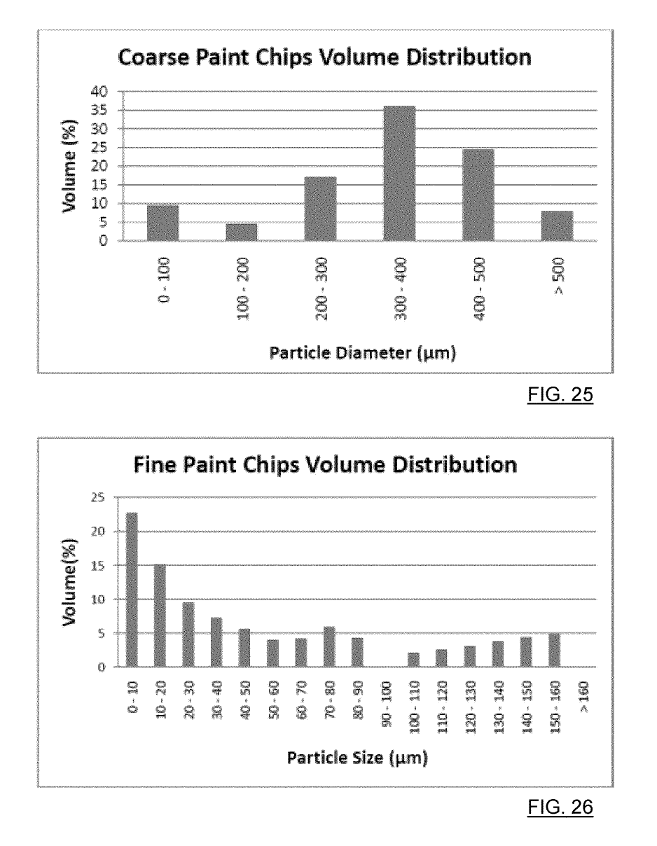

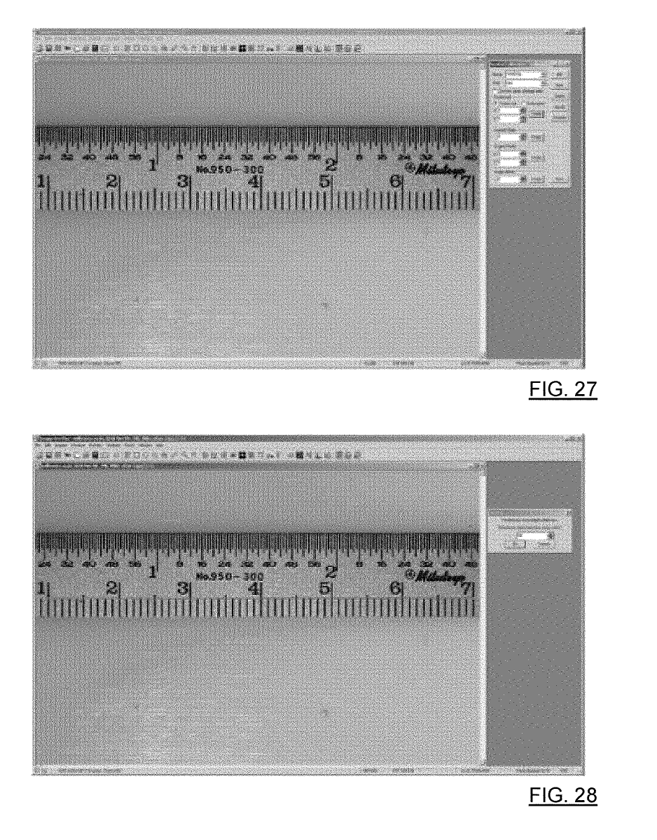

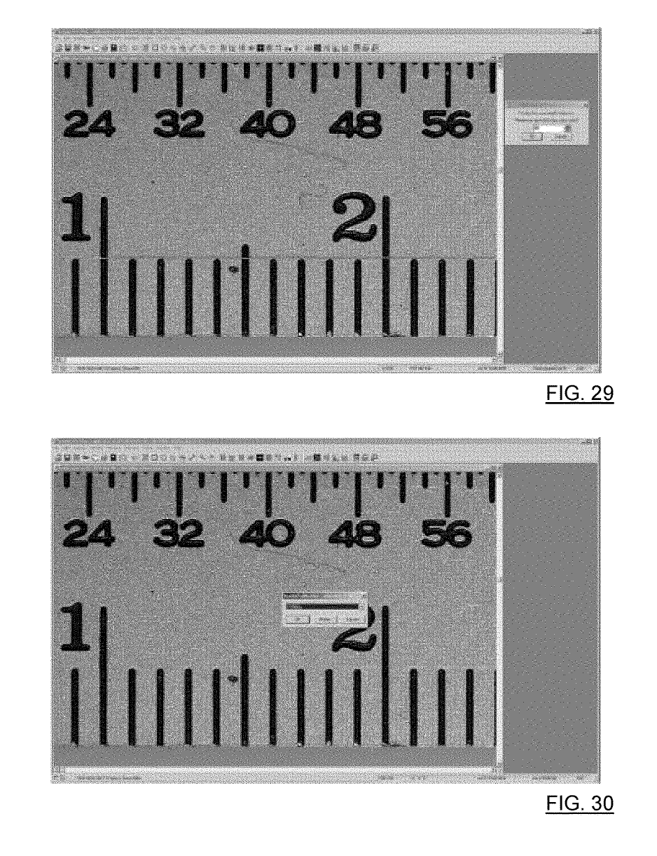

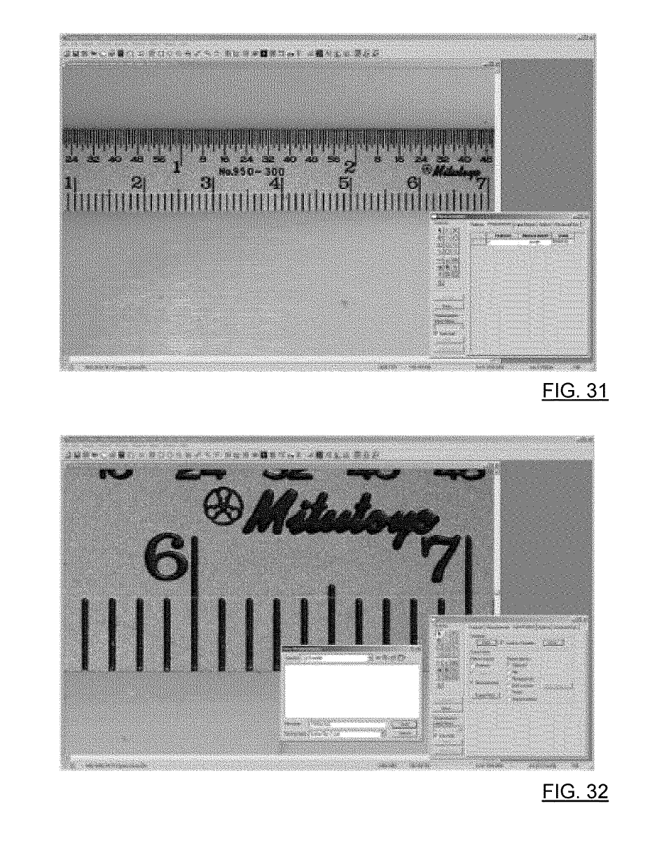

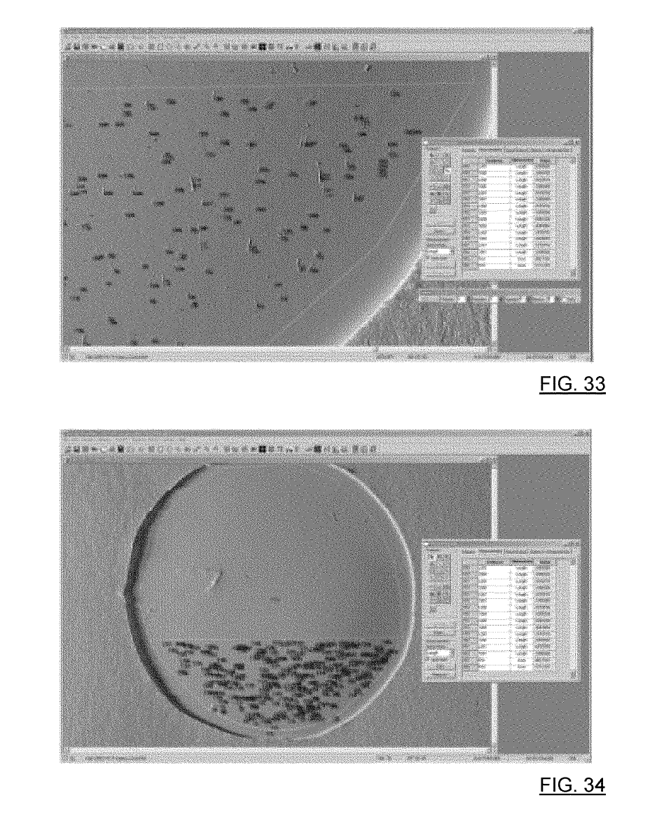

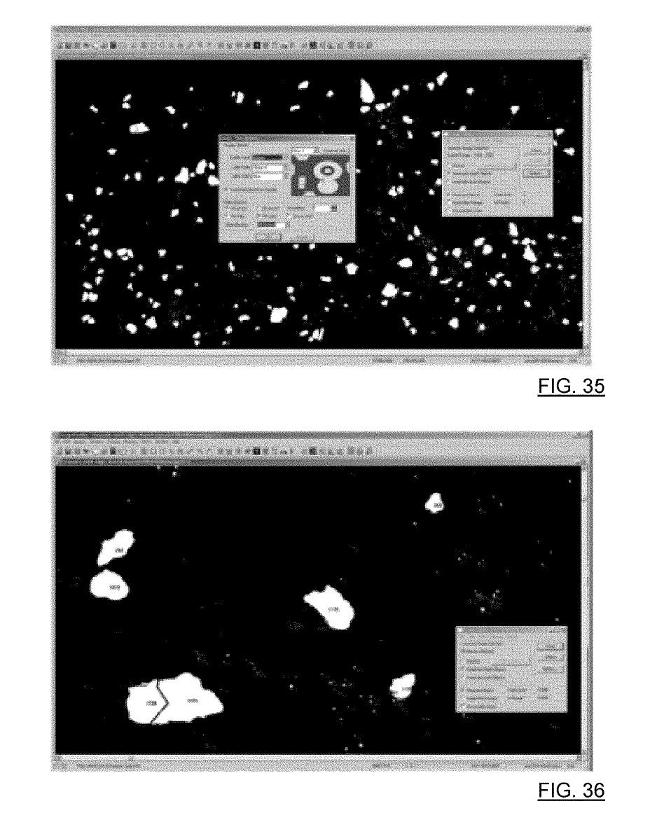

FIG. 6 shows an exemplary photographic setup for concrete particles;

FIG. 7 shows a sample support;

FIG. 8 is an image of concrete particles of 100 to 400 μm in diameter;

FIG. 9 is the image of concrete particles after thresholding;

FIG. 10 is an image of walnut shell flour;

FIG. 11 is an image of paint chips;

FIG. 12 is a graph showing fibre length distribution;

FIG. 13 is a graph showing fibre length time evolution;

FIG. 14 is a graph showing fibre diameter distribution;

FIG. 15 is a graph showing total fibre volume versus test time;

FIG. 16 is a graph showing Nukon fibre length distributions for different pre-treatments;

FIG. 17 is a graph showing Telisol fibre length distributions for different pre-treatments;

FIG. 18 is a graph showing Nukon mean and median fibre lengths for different pre-treatments;

FIG. 19 is a graph showing Telisol mean and median fibre lengths for different pre-treatments;

FIG. 20 is a graph showing Nukon fibre diameter frequency distribution;

FIG. 21 is a graph showing Telisol fibre diameter frequency distribution;

FIG. 22 is a graph showing volume distribution of concrete particles of 100 to 400 μm in diameter;

FIG. 23 is a graph showing volume distribution of concrete particles of 0 to 100 μm in diameter;

FIG. 24 is a graph showing walnut shell flour particle volume distribution;

FIG. 25 is a graph showing coarse paint chips volume distribution;

FIG. 26 is a graph showing fine paint chips volume distribution;

FIG. 27 is a software screenshot of spatial calibration set up;

FIG. 28 is a software screenshot of spatial calibration measurement;

FIG. 29 is a software screenshot of spatial calibration alignment;

FIG. 30 is a software screenshot of a spatial calibration check;

FIG. 31 is a software screenshot of a calibration check;

FIG. 32 is a software screenshot of a save measurements dialog box;

FIG. 33 is a software screenshot of fibre measurements;

FIG. 34 is a software screenshot of area measurements;

FIG. 35 is a software screenshot of count/size options; and

FIG. 36 is a software screenshot of count/size results.

### DETAILED DESCRIPTION

Various apparatuses or methods will be described below to provide an example of an embodiment of each claimed invention. No embodiment described below limits any claimed invention and any claimed invention may cover apparatuses and methods that differ from those described below. The claimed inventions are not limited to apparatuses and methods having all of the features of any one apparatus or method described below, or to features common to multiple or all of the apparatuses or methods described below. It is possible that an apparatus or method described below is not an embodiment of any claimed invention. Any invention disclosed in an apparatus or method described below that is not claimed in this document may be the subject matter of another protective instrument, for example, a continuing patent application, and the applicant(s), inventor(s) and/or owner(s) do not intend to abandon, disclaim or dedicate to the public any such invention by its disclosure in this document.

### 1 Photographic Techniques

This section describes the photographic techniques used for analyzing the fibre bypass grab samples and the fibre bypass test feedstock materials.

### 1.1 Photography of Fibre Bypass Filters

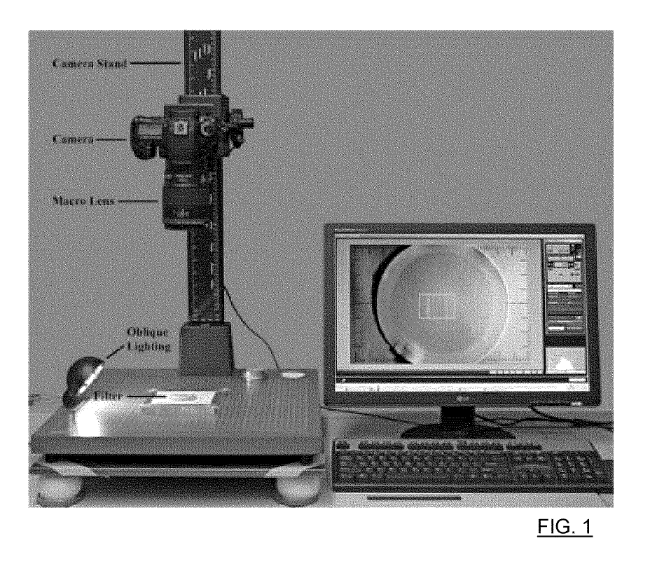

Referring to FIG. 1, high-resolution photographs taken with a macro lens are used to assess the bypass residue on a macroscopic basis (qualitative assessment of amount and type of debris over whole filter) and at higher resolution to determine fibre counts and total length. The camera used is a Canon EOS 7D™ digital SLR equipped with a Canon EF™ 100 mm, f/2.8 L macro lens. The camera is mounted on a Kaiser RS1™ camera stand having an RA1 arm using the ¼-20 tripod thread in bottom of the camera.

In order to ensure that the image is in focus across the entire frame, the camera's sensor plane may be made parallel to the base of the camera stand (or the specimen holder) using a two-axis bubble level. First, a photograph of the bubble level reading for the camera stand's base is made and displayed on the camera's LCD screen. Next, the level is placed on the LCD screen of the camera and the camera is adjusted until the bubble locations correspond to the photograph for the base.

The height of the camera is adjusted until the filter image is framed as it appears on the monitor screen in FIG. 1. This ensures the resolution is optimum.

The camera's operation is controlled by Canon EOS Utility™ software (version 2.8.1.0). The software allows remote control over most of the camera's settings, focus adjustment and the shutter trigger. The preview screen, which may be seen on the left of the monitor in FIG. 1, shows the image on the camera's sensor, allowing precise framing, focussing and, exposure control. Focussing is accomplished using the controls provided by the software.

While photographing fibre bypass filters, the camera should be in manual exposure mode with the following settings: f-ratio f/8; ISO 100; Al focus; Automatic white balance; and Metering Mode set to Evaluative. The macro lens should have image stabilization turned OFF and autofocus turned ON. The correct shutter speed may be determined by trial and error, since the brightness of the sample will vary. The captured image may be overexposed compared to what is shown on the computer monitor video image. Several test shots may be required to find the correct shutter speed that gives a properly exposed image.

At the correct shutter speed for a captured image, the image on the monitor screen will be too dark to frame and focus the next filter. To get the right exposure on the computer monitor video image, the gain on the sensor may be adjusted by changing the ISO setting temporarily.

Filter lighting should be oblique (see FIG. 1). This creates long shadows for the fibres, which makes them more easily identified and measured. A 20 watt quartz halogen bulb has been used to good effect. The reflector in the lamp housing was painted black so that the shadows produced would have a sharper outline.

To determine the scale of the filter images, an image of a steel ruler is made at the end of a filter series. The lens focus may not be changed for this image or the magnification will not be the same as the filter images. The camera may be moved using the crank on the camera stand (or the ruler's height may be adjusted) until the ruler is in focus.

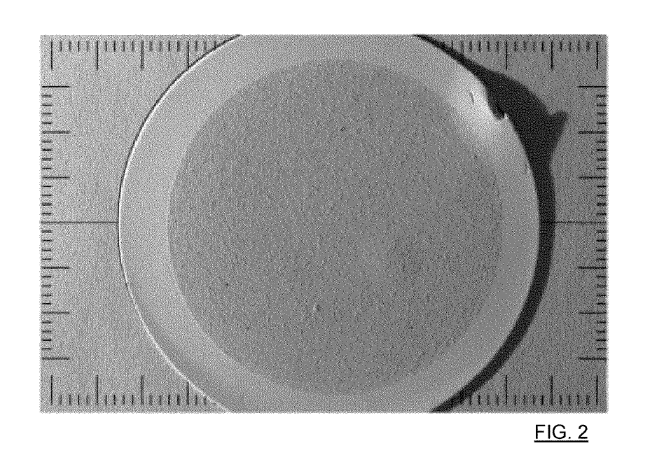

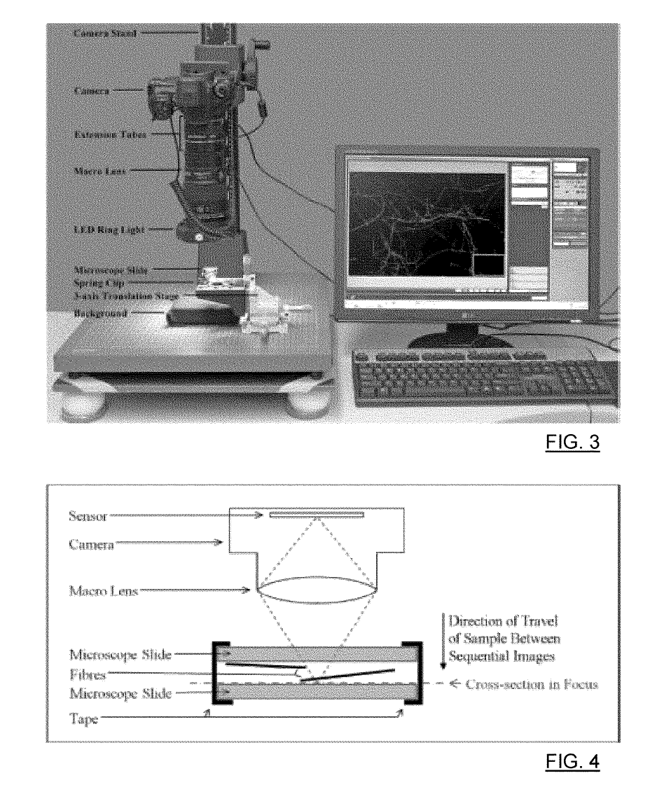

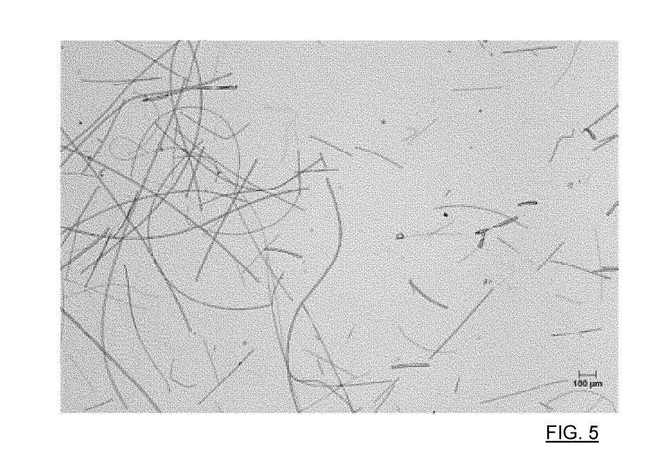

An example of an image obtained with this setup is shown in FIG. 2. Note the shadows cast by the oblique lighting. The resolution of this image is about 12 microns per pixel.

### 1.2 Characterization of Insulation Fibres by Photographic Techniques

To characterize new insulation types by fibre diameter and length, two techniques referred to as focus stacking and image stitching may be used, as described in further detail below.

### 1.2.1 Photography of Insulation Fibres for Fibre Diameter Measurement

Samples of different types of fibreglass are prepared for photography by using tweezers to gently pull a small number of fibres from the mat of fibres provided and placing them on a microscope slide. This produces a small group of highly entangled fibres which may be gently torn apart using thin, sharpened metal rods. Complete separation may not be achieved in a reasonable time but it is satisfactory if the fibres do not remain as dense clumps. The fibres should be arrayed over the central 75% of a slide and then covered with a second slide to hold them in place. The two slides are then fastened together with tape at each end of the slide and the specimen ID written on the tape. It is advisable to wear a dust mask while doing this to prevent inhalation of the fibres and to prevent exhalation from blowing the fibres away.

The photographic setup for insulation fibre photography is shown in FIG. 3. The setup uses the same camera as described in section 1.1 above but the lens used is a Canon MP-E™ 65mm macro lens, at 5× magnification, with a set of 3 Kenko™ extension tubes to increase the magnification. The oblique lighting is replaced by an LED ring light. A dark background is shown but a white background also works well. The microscope slide is fastened to the three-axis translation stage with a spring clip so that the area to be photographed overhangs the horizontal platform. For clarity, the clamping system used to secure the translation stage to the base of the camera stand is not shown.

The lens f-ratio should be set to 2.8 for the sharpest images. The lens magnification is set at the maximum 5×. The other settings are the same as described in section 1.1 above. The procedure for determining the shutter speed is also the same. The vertical axis (Z) of the translation stage should be within 3 to 4 turns of the top of its travel. Rough focussing is done with the crank on the camera mount. The camera should be brought as close to the microscope slide as possible, using the hand crank on the camera stand, and then raised slowly until the fibres are approximately in focus. The vertical axis (Z) of the translation stage is then used to fine focus onto the top of the lower microscope slide (see chain-dot horizontal line in FIG. 4).

The horizontal (X-Y) motions of the stage are used to select an area of the slide with reasonably well separated fibres. The focus is adjusted using the vertical axis translation control until one or two fibres just come into focus. Now lower the microscope slide until one or two fibres just come into focus. These are fibres that are on top of the lower slide. Note that there are many fibres that will be out of focus that are suspended between the upper and lower slides. It will not be possible to obtain an image with all of the fibres in focus at the same time. A technique called focus stacking is used to obtain an image with all the fibres in focus.

Focus stacking involves taking a series of images, each focussed along a particular cross-section or plane of the sample, and each focused at progressively closer distances, and then combining them so that only the focussed portion of each image is included in the final composite image. The focus of the lens should not be changed while doing this or the image magnification will be changed. Instead, either the camera or the object should be moved. In this case, the vertical axis of the translation stage is used to move the fibres between the microscope slides through the plane where the macro lens is focussed. (See FIG. 4.)

Due to the way focus stacking algorithms work, it is important the sequence of image numbers increase with decreasing distance from the camera. It is recommended that the vertical translation stage be adjusted in increments of 0.001 inch until all the fibres visible on the computer monitor have gone from unfocussed to focussed, and back to unfocussed again. A focus stacked image is shown in FIG. 5. The resolution of the original image is 0.64 microns per pixel. Note that the image was taken against a white background.

This series of photographs transformed into a single image using CombineZM™ software. The lengths and diameters of the fibres are then able to be measured using Image-Pro Plus™ software, which is described in further detail in Appendix A. The use of CombineZM™ and Image-Pro Plus™ software is provided by way of example only, and are intended to be illustrative and non-limiting.

To calibrate the scale of the image(s), a photograph of a steel ruler may be taken without changing the focus of the lens.

### 1.2.2 Photography of Insulation Fibres for Fibre Length Measurement

In order to measure the length of insulations fibres, and obtain a fibre length distribution, several overlapping, focus stacked images are made and stitched together using Photoshop™ or another suitable image processing program. This is referred to as image stitching. All camera settings are the same as in the previous section. Images should overlap sufficiently to allow good alignment. Since there will be some distortion near the edge of each image, it is helpful to align the layers in Photoshop™ using the difference mixing mode.

To calibrate the scale of the image(s), a photograph of a steel ruler may be taken without changing the focus of the lens.

### 1.3 Characterization of Particulate Debris by Photographic Techniques

Fibre bypass in a full debris load test may include particulate debris. Thus, it is desirable to characterize the particulate debris that is added.

### 1.3.1 Photography of Concrete Particles

Two size ranges of concrete particles are used for strainer testing: 100 to 400 μm; and <100 μm. The raw material used to prepare these concrete particles was obtained from the Dalhousie University Minerals Engineering Centre. Concrete particles are supplied in two size ranges: 1.0 mm to 0.1 mm; and 0.1 mm to 0.075 mm.

The 100 to 400 micron particles are prepared by sieving the 1.0 mm to 0.1 mm material using sieves having 0.297 mm and 0.106 mm openings. The <100 micron particles are prepared by sieving the 0.1 mm to 0.075 mm material using a sieve having 0.106 mm openings. The samples are stored in sealed plastic bags after preparation.

Representative samples for particle size distribution measurements are obtained by first gently agitating the sealed plastic bags to ensure that the particles are thoroughly mixed. Next, a small sample (1 to 2 g) is taken and placed in a 50 mL plastic centrifuge tube. The process of agitation and sampling is repeated until a total of 30 to 40 g of material has been obtained. This method minimizes the effects of particle segregation which may occur when samples have been stored for an extended period and is applicable to many types of materials.

The setup for concrete particle photography is shown in FIG. 6. The lens is a Canon MP-E™ 65 mm, f/2.8, 1−5× macro lens. Coarse focussing is done with the crank on the camera stand and fine focussing with the vertical axis of the translation stage. Three Kenko™ extension tubes (shown in the image) with a total length of 68 mm may be used to increase the resolution to about 0.65 microns per pixel but are usually not needed, since the resolution without them is 0.856 microns per pixel.

Details of the sample support are shown in FIG. 7. The conical reflector provides back illumination for the particles being photographed. This eliminates dark edges, which may confuse the measurement software. The felt-lined interior of the reflector support and the felt background prevent light from the ring light from being reflected back to the camera, thus keeping the background of the photograph uniformly dark.

The method for determining shutter speed is the same as that for filter photography. The camera should be in manual exposure mode with the following settings: f-ratio f12.8; Magnification 5× (fully extended); ISO 100; Automatic white balance; and Metering Mode set to Evaluative.

A very small quantity of the concrete particles to be measured is sprinkled on the Petri dish which is then placed on the sample support. Three hundred to four hundred particles are sufficient to obtain a good particle size distribution. Ideally the particles should not touch each other but this will rarely be achieved. A series of images for focus stacking is then made using the same procedure as that for insulation fibres. The images are then stacked using CombineZM™ software. A typical image of 100 to 400 micron concrete particles is shown in FIG. 8. Note that many particles are almost black in colour but do have a thin light border due to the illumination from the reflector shown in FIG. 9.

The Image-Pro Plus™ measuring software expects light particles against a dark background. The image in FIG. 8 would produce erroneous results without some processing. Photoshop™ is used to threshold the image and paint in any remaining dark areas prior to measuring the image. The result is shown in FIG. 9.

To calibrate the scale of the image(s), a photograph of a steel ruler may be taken without changing the focus of the lens.

### 1.3.2 Photography of Walnut Shell Flour Particles

The walnut shell flour used for strainer head loss tests and possibly in future fibre bypass tests is generally supplied by Industrial Supply and obtained through Canada Bearings. The material is listed as −325 mesh. The specification states that, by weight, the particle size distribution is as follows:

<7.41 μm<10%; and

>44.39 μm<10%.

The material is sampled by removing 1 to 2 gram samples from various locations in the storage container and placing them into a 50 mL plastic centrifuge tube until 40 or 50 g have been collected.

The photographic set up, techniques and camera settings are identical to those for photographing concrete particles as described in section 1.3.1 above.

An image of walnut shell flour is shown in FIG. 10. Prior to measuring the image with ImagePro Plus™, the image may be processed by thresholding, as described for concrete particles.

To calibrate the scale of the image(s), a photograph of a steel ruler may be taken without changing the focus of the lens.

### 1.3.3 Photography of Paint Chips

Paint chips are prepared as follows:
Spray Epoxy (4500) onto large (4 ft×8 ft) plastic sheets in the AECL-CRL Paint Shop.
Spray four layers of paint to result in an overall layer thickness of 101 to 203 μm.
Allow the painted sheets to cure for a minimum of 1 day, after which time they may be rolled loosely and stored at room temperature for further curing. The total minimum cure time from spraying to removal from the plastic sheets is 5 days.
Separate the cured paint from the plastic sheets as large flakes.
Manually break the large flakes into smaller flakes of less than 1 in. (25 mm) diameter,
Break the flakes into chips of less than 0.5 mm diameter by adding the flakes to a blender.
Sift the flakes through a sieve with 400 μm openings.
Sift the flakes though a sieve with 100 μm openings.
Collect and label coarse paint chips on top of the 100 μm sieve (size: 100 to 400 μm)
Collect and label fine paint chips through the 100 μm sieve (size: less than 100 μm)

Paint chips could also be prepared by dry vibratory milling. Two size categories are usually generated. One size ranges from 100 to 400 μm (coarse) and another less than 100 μm (fine).

The photographic set up, techniques and camera settings are identical to those for photographing concrete particles as described in section 1.3.1 above.

A typical image of paint chips prepared in the manner described is shown in FIG. 11. As before, the image may be processed by thresholding prior to analysis with ImagePro Plus™.

To calibrate the scale of the image(s), a photograph of a steel ruler may be taken without changing the focus of the lens.

### 2 Analysis Results

Photographs are analyzed using ImagePro Plus™ software, which is described in Appendix A.

### 2.1 Fibre Length Distributions

An example of the fibre length distribution for a typical fibre bypass test is shown in FIG. 12. A total of 582 fibres, with a total length of 83.6 mm, were measured. This represented the fibres on 29.6% of the filter area and the results were scaled to 100% to determine the total fibre length for the entire filter.

When a series of filters have been analyzed, it is informative to look at the evolution of the fibre length distribution with time. FIG. 13 shows the result for a test where periodic debris additions were made at the times indicated by the vertical lines. In FIG. 13, the number of fibres in each size range has been expressed as a multiple or a fraction of the number of fibres in the smallest size range (0 to 0.1 mm, horizontal line with diamond markers).

### 2.2 Fibre Diameter Distributions

A typical fibre diameter distribution for Nukon fibre glass insulation is shown in FIG. 14. A total of 544 fibres were measured. Mean fibre diameter was 0.0066 mm (average diameter).

### 2.3 Fibre Volume Distributions

In order to calculate the volume distribution of the bypassed fibres, it is necessary to know both the diameter and length of each fibre. This is practical when only a few samples are to be measured but becomes time consuming when a large number of samples are involved.

The total fibre volume of a sample is also a quantity of interest. Once again, it is time consuming to measure the diameter and length of each fibre. An alternative method involves making a one time determination of a fibre diameter which represents the diameter of a fibre at the average of the fibre volume distribution. Using this average diameter, D_{avg}, it is then possible to calculate a value for the total fibre volume of any sample (derived from the same feedstock and subjected to the same test conditions) based solely on the total fibre length of the sample.

To calculate the volume-weighted D_{avg}, the individual fibre diameters (Dᵢ) of N fibres of a representative sample of fibreglass are measured. Four to five hundred fibres are measured. The cross-sectional areas of all the fibres are then summed (A) and D_{avg} calculated.

$$A = \frac{π}{4} {\sum }_{i = 1}^{N} {D}_{i}^{2}$$

$${D}_{avg} = \sqrt{\frac{4}{π} \cdot \frac{A}{N}}$$

Using the same data that was used to plot FIG. 13 and FIG. 14, the total fibre volume was calculated (as described above) for each filter and plotted as a function of the test time (see FIG. 15). Note that after the final debris addition the fall-off in fibre volume is exponential.

### 2.4 Effect of Pre-Processing on Fibre Lengths

Prior to using fibreglass as part of the debris load for a strainer test, the fibres are pre-treated in order to simulate the effects of a steam jet on the pipe insulation during a pipe break. Both Nukon and Telisol fibreglass were subjected to four different pre-treatments and the resulting fibre length distributions measured photographically using ImagePro Plus™ software. The results are shown in FIG. 16 and FIG. 17. The changes in the mean and median fibre lengths are shown in FIG. 18 and FIG. 19.

From the figures it may be seen that shredding decreases overall fibre length to a greater degree than spraying, and that overall fibre length decreases with increased levels of processing.

### 2.5 Nukon and Telisol Fibre Diameter Comparison

The fibre diameter distributions for Nukon and Telisol were measured photographically using ImagePro Plus™ software. Both varieties had been aged, shredded and sprayed prior to measurement. The results are shown in FIG. 20 and FIG. 21. Telisol has 62.4% of fibres with a diameter of less than 5 microns, which is about twice as many as Nukon at 33.5% of fibres. This may partially account for the perception that Telisol “feels softer” than Nukon.

### 2.6 Concrete Particle Size Distributions

The size distribution of the concrete particles used for a test is measured for two reasons: verification of the supplier's material specification and confirming that the material preparation method used produces particle size ranges meeting the strainer test plan specification.

Sieves having 0.297 mm and 0.106 mm openings are used to separate the 100 μm to 400 μm concrete particles from the 1000 μm to 100 μm material provided by the supplier. The sieving operation is carried out by an electric shaker. As long as the motion of the sieve is in the plane of the sieve, the particles should lay flat and the largest dimension of the particles passing through will be approximately equal to the diagonal of the sieve spacing.

The volume (mass) distribution for 100 to 400 μm concrete particles is shown in FIG. 22. About 12% of the volume of the particles is due to those that are larger than 400 μm. These large particles comprise only 0.023% of all the particles. The 297 μm sieve openings have a diagonal dimension of about 420 μm and will allow particles with this dimension through only if the particle is not laying flat when it is in contact with the opening. Agitation of the sieve during screening may cause particles to occasionally be upended, allowing them to pass through the sieve.

About 2% of the volume of the particles is due to those that are smaller than 100 μm. These small particles comprise 98% of all the particles. Since sieving the <400 μm particles with the 106 micron sieve should have eliminated this size range, either the sieving was not continued for a sufficient length of time or they are a result of self-comminution, where particles rubbing against each other create smaller particles.

The volume (mass) distribution for the 0 to 100 μm concrete particles is shown in FIG. 23. As was seen for the 100 to 400 μm particles, the upper limit for the particles size is approximately equal to the diagonal of the sieve opening, which for the 106 μm sieve is 150 μm.

### 2.7 Walnut Shell Flour Size Distribution

The volume (mass) distribution for −325 mesh walnut shell flour is shown in FIG. 24. The volume distribution agrees well with the manufacturer's specified distribution of less than 10% smaller than 7.41 μm and less than 10% larger than 44.39 μm.

### 2.8 Paint Chips Size Distributions

The volume (mass) distribution of the paint chips are shown in FIG. 25 and FIG. 26.

### 3 Conclusions

The methods disclosed herein represent a robust set of techniques for the analysis of fibre bypass strainer test samples and the characterization of the properties of strainer bypass test feedstock materials. The methods disclosed herein may be used to characterize the size distribution of fibres or particulates as small as 2 μm. The methods disclosed herein may be useful in quantifying long-term fibre bypass more quickly and using less costly equipment than the previously used SEM technology. It also may improve the measurement accuracy as larger filter areas may be analyzed.

While the present disclosure emphasizes fibre and particulate quantification in the context of nuclear power plant safety, techniques disclosed herein have the potential for use for a wide range of scientific and research activities, and may also be applicable in a wide range of industries (e.g., pulp and paper industry), where counting and measuring fibres are required. It may also be used for forensic applications to visually identify very small stains, particles, fibres and objects.

While the above description provides examples of one or more methods or apparatuses, it will be appreciated that other methods or apparatuses may be within the scope of the accompanying claims.

### Appendix A

### Image Analysis Software

Measurements of particles and fibres are made using ImagePro Plus™software for Windows™ by Media Cybernetics, Inc. The version used by the inventors was 4.5.1.22.

### 1 Fibre Bypass Filter Analysis

### 1.1 Step 1—Image Scale Calibration

1. After the filter has been photographed and a calibration image of a steel ruler has been made, open the ImagePro Plus™ software.
2. Open the calibration image—File>Open.
3. Measure the calibration image—Measure>Calibration>Spatial (see FIG. 27).
4. Click the “New” button.
5. In the “Name” box, type a suitable name. Suggested form is YYMMDD-letter, e.g., 110923a for 2011, September 29 and a letter a-z where “a” is the first calibration of that day, “b” is the second, etc.
6. In the “Unit” box select “mm”.
7. Click the “Image” button.
8. Place the cursor on the box on the ends of the white line and click and drag the endpoints of the line to the start and end points on the image (see FIG. 28.). In this case, move the box on the left side of the line to the LEFT side of the ruler line corresponding to 1 cm, then move the box on the right side of the line to the LEFT side of the ruler line corresponding to 7 cm.
9. At this point it is desirable to zoom in on the ends of the line in order to precisely align them with the ruler marks. Right click on the image and select Zoom>Zoom 100%.
10. Scroll to the line's left endpoint. Place the cursor over the line, then left click and drag it until the end is precisely aligned with the LEFT edge of ruler line (1 cm in this case) and it just touches the mm ruler lines. (See FIG. 29) Since the ruler may not be precisely aligned with the edge of the photograph, the line should be aligned with the edge of the ruler to ensure a precise calibration. Aligning each end of the line with the tops of the mm ruler marks achieves this alignment.
11. Scroll to the right endpoint of the white line. Place the cursor on the box on the right end of the line, then click and drag it until it is precisely aligned with the LEFT edge of the ruler line (7 cm in this case) and is just touching the tops of the 1 mm ruler lines. It will likely be necessary to go back and realign the left end of the white line with the tops of the 1 mm ruler lines again. Repeat as necessary.
12. In the Scaling dialog box (on the right in FIG. 29) use the up and down arrows to change the reference units to the correct value (60 mm in this case).
13. Press the “OK” button and press “OK” again.
14. Press the Spatial Calibration Icon (yellow caliper, 8th icon from right in tool bar). The name in the box should correspond to the name you gave the spatial calibration you just created (in this example it is 110923a). If not, select it from the drop down box. (See FIG. 30.) Click the “OK” button.
15. Now save the spatial calibration in the same folder as the image of the rule you used to create it. Select—Measure>Calibration>Save Active . . . The filename should be the same as the spatial calibration name.
16. Now check the accuracy of the spatial calibration by measuring the distance between the 1 cm and 7 cm ruler lines. From the tool bar, select Measure>Measurements . . . .
17. Select the Measurements tab.
18. Select the Straight Line tool from the Features panel.
19. Right click on the ruler image and zoom until the entire ruler is in view.
20. Click on the left side of the 1 cm line on the image of the ruler and drag the line until it is on the left side of the 7 cm line on the image of the rule. (See horizontal line in FIG. 31.) Note that in the Measurements panel, a Feature (L1) has appeared with Measurement (Length) and Value (59.84315). The horizontal line now has a blue label “L1” as well.
21. Zoom in on the left end of the yellow line.
22. Choose the Select tool from the Features panel in the Measurements panel.
23. Click on the yellow line and a white box will appear on each end. The cursor will change to a hand when hovering over the line.
24. Click and drag the line until the left end is aligned with the LEFT side of the 1cm line on the ruler and just touching the tops of the 1 mm ruler lines. Clicking elsewhere on the image will cancel the boxes on the ends of the line, allowing the alignment to be checked precisely.
25. Repeat Step 17 for the right end of the yellow line, aligning it with the LEFT edge of the 7 cm ruler line and the tops of the mm ruler lines.
26. When the line is precisely aligned, the length measurement for L1 will change to 60.
27. Select the Input/Output tab in the Measurements panel.
28. Click the Save button. The Save Measurements panel will open. Navigate to the folder where the calibration image is saved.
29. From the Save as type window, select Outline Files (*.out).
30. For the file name, use the name of the calibration plus CAL, in this case 110923a-CAL. (See FIG. 32.)
31. Click Save.
32. Close the calibration image.

### 1.2 Step 2—Fibre Length Measurement

1. Open the image of the filter to be measured—File>Open.
2. Open the Measurements panel—Measure>Measurements.
3. Select the Measurements tab.
4. Select the Trace tool.
5. Zoom in to 100% on the filter image.
6. Left click on the endpoint of a fibre and move the cursor along the fibre, left clicking as required to follow the shape of the fibre. Right click when the other endpoint is reached. (See FIG. 33) The fibre trace features are prefixed with a “T”. In this example, the fibre diameters were also measured with the Line tool, and are prefixed with an “L”.
7. Continue until 200 to 300 fibres have been measured. It is best to start measuring at the right hand edge of a filter, since the feature labels tend to obscure features to their right. Since a filter may contain many hundreds of fibres, start at the top or bottom of a filter and work right to left, then move back to the right hand edge and move vertically to start another row. Finish each row you start. Any fibre which is off the edge of the photo is not measured. Any fibre which intrudes into a row you are doing is measured.
8. If the trace of a fibre is incomplete, choose the Features tab in the Measurements panel, then select the Select tool. Click on the feature number and the line corresponding to the trace will acquire handles with which the feature may be manipulated. To delete the trace, click on the black X in the features panel. Do not click the red X as this deletes all features.
9. Once 200 to 300 fibres have been measured, the measured area of the filter (PG1) and the total active area of the filter (PG2) are measured. Select the Polygon tool from the features panel.
10. Trace the area of the filter which has been measured, then trace the total active area of the filter (see FIG. 34). This completes the fibre measurements.
11. Save the measurements as a *.out file. Select the Input/Output tab in the Measurements panel. Click the Save button. The Save Measurements panel will open. Navigate to the folder where the filter image is saved (similar to FIG. 32).
12. From the Save as type window, select Outline Files (*.out).
13. For the file name, use the name of the filter.
14. Click Save.
15. In the Input/Output tab of the Measurements panel, under the Export data section, select Data to Output>Measurements and Output Data to>Clipboard.
16. Open Excel and the appropriate data analysis template and paste the data into the spreadsheet.
17. Save the analysis template spreadsheet with an appropriate name (filter name).
18. Close ImagePro Plus™.

The same techniques used for measuring fibres on filters may be used for measurement of fibres only.

### 2 Particle Measurements

1. For the measurement of particles, such as walnut shell flour and concrete, the procedure from section 1.1 is followed first.
2. Open ImagePro Plus™ software.
3. Open the image to be measured File>Open. The image should be white objects on a black background. The software will not measure images that do not have a high contrast level between the particles and the background. This will require processing of the image by a suitable image manipulation program. Also, particles that touch or overlap may be separated by the background colour (black). Although there are routines in the ImagePro Plus™ software that may attempt to automatically detect and separate overlapping particles, the process may be somewhat overzealous and may be easily fooled.
4. From the Menu Bar select Measure>Calibration>Open . . . .
5. Navigate to the folder with the spatial calibration measured in Step 1 and open the *.cal file.
6. From the toolbar, select Measure>Count/Size . . . .
7. Select Automatic Bright Objects and Measure Objects (see FIG. 35).
8. In the Count/Size panel menu bar, select Measure>Select Measurements.
9. Click Select None.
10. In the Measurements window, scroll down and select Diameter (max), Diameter (mean) and diameter (min).
11. Click OK.
12. In the Count/Size panel, click Options. Select the following options:

Outline Style—Outline;

Label Style—Object #,

Dark background on Sample—checked;

4-connect;

Fill Holes—checked;

Smoothing=0; and

Clean Borders=All Borders.
13. Click OK.
14. In the Count/Size panel, click Count. The image changes and each object is now outlined and has a feature number associated with it. A 50% zoomed image is shown in FIG. 36. Note the manual separation of the two large particles 1736 and 1695.
15. In the Count/Size panel menu bar, select File>Save Outlines. Navigate to the folder where the particle image is stored. Choose an appropriate filename and click Save.
16. In the Count/Size panel menu bar, select File>Data to Clipboard.
17. Open the appropriate analysis template in Excel and paste the data into the spreadsheet.
18. Save the analysis spreadsheet with an appropriate filename in the same folder as the particle image and the outlines file.
19. Close Excel.
20. Close ImagePro Plus™.

This procedure is also used to measure paint chips.

1. A method of quantifying debris content of a water and debris mixture, comprising:
obtaining at least one image of a sample of the water and debris mixture; and
analyzing the image to quantify the debris content.

2. The method of claim 1, wherein the step of obtaining comprises obtaining a plurality of images of the sample.

3. The method of claim 2, wherein the step of obtaining comprises focussing the plurality of images at different cross-sections of the sample.

4. The method of claim 3, further comprising combining the plurality of images of the sample to generate a composite image comprising focussed portions of each image.

5. The method of claim 4, wherein the step of obtaining comprises using a camera to obtain the plurality of images of the sample, and the step of obtaining comprises varying a distance between the camera and the sample to obtain the plurality of images of the sample.

6. The method of claim 5, wherein the step of obtaining comprises progressively decreasing the distance between the camera and the sample.

7. The method of claim 1, wherein the step of analyzing comprises using image analysis software.

8. The method of claim 7, wherein the step of analyzing comprises using the image analysis software to measure fibre diameter for a plurality of fibres of the mixture.

9. The method of claim 8, wherein the step of analyzing comprises calculating an average diameter.

10. The method of claim 9, wherein the step of analyzing comprises using the image analysis software to measure fibre length for a plurality of fibres of the mixture.

11. The method of claim 10, wherein the step of analyzing comprises calculating a total fibre length.

12. The method of claim 11, wherein the step of analyzing comprises calculating a total fibre volume for the plurality of fibres.

13. The method of claim 7, wherein the step of analyzing comprises using the image analysis software to measure particle diameter for a plurality of particles of the mixture.

14. The method of claim 1, wherein the step of obtaining comprises using a digital camera.

15. The method of claim 1, further comprising, prior to the step of analyzing, thresholding the at least one image.

16. (canceled)

17. The method of claim 1, wherein the step of analyzing comprises:
measuring fibre diameter for a plurality of fibres of the mixture;
calculating an average diameter;
measuring fibre length for the plurality of fibres of the mixture;
calculating a total fibre length; and
calculating a total fibre volume for the plurality of fibres based on the average diameter and the total fibre length.

18. The method of claim 6, wherein the step of analyzing comprises measuring fibre diameter for a plurality of fibres of the mixture.

19. The method of claim 6, wherein the step of analyzing comprises measuring fibre length for a plurality of fibres of the mixture.

20. The method of claim 6, wherein the step of analyzing comprises measuring particle diameter for a plurality of particles of the mixture.

21. A method, comprising:
obtaining a plurality of images of a sample of a water and debris mixture;
combining the plurality of images of the sample to generate a composite image comprising focussed portions of each image; and
analyzing the composite image to quantify debris content,
wherein the step of analyzing comprises at least one of
measuring fibre diameter for a plurality of fibres of the mixture,
measuring fibre length for a plurality of fibres of the mixture, and
measuring particle diameter for a plurality of particles of the mixture.
